(19) United States
(12) Patent Application Publication
(10) Pub. No.: US 2016/0029929 A1
(43) Pub. Date: Feb. 4, 2016

(54) Posture monitor and correction device

(71) Applicants: Mark John Worthen, Cloverdale, CA (US); Sheila Ann O'Halloran, Cloverdale, CA (US)

(72) Inventors: Mark John Worthen, Cloverdale, CA (US); Sheila Ann O'Halloran, Cloverdale, CA (US)

(21) Appl. No.: 13/986,383

(22) Filed: Apr. 25, 2013

Related U.S. Application Data
(60) Provisional application No. 61/687,841, filed on May 1, 2012.

(51) Int. Cl. A61B 5/11; A61B 5/00

(52) U.S. Cl. CPC A61B 5/1116; A61B 5/742; A61B 5/7405; A61B 5/7455; A61B 5/6898; A61B 5/6803; A61B 5/6804; A61B 5/72

(57) ABSTRACT

An apparatus for monitoring an individual's posture and identifying incorrect deviations includes a level sensor and a control and data unit attached to the apparatus. The apparatus is worn by a user and may be attached to or included as part of a hat, cap, head band, belt, eyeglasses, sunglasses, garment or article of clothing. The control and data unit records deviations away from a predetermined normal posture of the user in a given period of time and either communicates the deviations to the user in real time for immediate postural correction or records the deviations for later downloading or transmission and analysis. A preferred embodiment of the apparatus includes a cap with LED lights that indicate deviations away from ideal posture that exceed a tolerance limit to the user as they occur.

This patent application claims the Benefit of the Date of Priority of Provisional Patent Application Ser. No. 61/687,841 that was filed on May 1, 2012 by the same inventors, Mark John Worthen and Sheila Ann O'Halloran, entitled “Posture Monitor and Correction Device aka “The blinkin hat” or “The smart hat”.

### RESERVATION OF RIGHTS

A portion of the disclosure of this patent document contains material which is subject to intellectual property rights such as but not limited to copyright, trademark, and/or trade dress protection. The owner has no objection to the facsimile reproduction by anyone of the patent document or the patent disclosure as it appears in the Patent and Trademark Office patent files or records but otherwise reserves all rights whatsoever.

### BACKGROUND OF THE INVENTION

1. Field of the Invention

The present invention, in general, relates to posture and, more particularly, to a device for verifying correct posture in real time.

It is well known that people suffer from poor posture. Aside from detracting from appearance poor posture affects long-term muscular-skeletal health and functioning, such as range of motion. Poor posture can also subject a person to increased physical stress which, over the course of time, can lead to muscular-skeletal strains, sprains and more serious injuries, such as spinal disc injuries or other damage, such as tearing of tendons, damage to ligaments, etc.

The health and comfort of humans are enhanced by maintaining proper posture in all aspects of daily living. Musculoskeletal Disorders (MSDs) include sprains and strains. These MSDs are costly to individuals, their families, and the organizations which employ them. Studies clearly show that maintaining correct postures result in fewer injuries. Studies show that proper posture: 1) keeps bones and joints in the correct alignment so that muscles are used properly, 2) helps decrease the abnormal wearing of joint surfaces, 3) decreases the stress on the ligaments holding the joints of the spine together, 4) prevents the spine from becoming fixed in abnormal positions, 5) prevents fatigue and pain because muscles are being used more efficiently, allowing the body to use less energy, thereby increasing postural performance 6) prevents backaches and muscular pain, and 7) contributes to good appearance.

It has been established that, given the symmetry of the body about the saggital and coronal planes, a key element of posture is the inclination and lateral movement of the head in these planes, statically and dynamically with body movement, along with the necessary linear elongation of the neck and spine required for truly good posture. There is an optimal range of head tilt within which the weight of the head tends to be balanced and thus minimizes the amount of force required from supporting muscles, ligaments and bones. Generally, individuals whose normal head positioning is held within this optimal range, unbalancing of the weight of the head upsets the equilibrium, and, if continued over a length of time, generally leads to symptoms of muscle and ligamentous strain/sprain and potential, discomfort or dysfunction along with the resultant detriment to appearance and self-esteem.

As a result of several studies, including the observation of many material or manual handlers, at-desk workers and personal technical device users, it was shown that a person's head position is the leading indicator of entire body posture. By managing head tilt a person's overall posture can be significantly improved, protecting them from MSDs or MSD injury. For example, a computer operator's back pain or wrist pain may be caused when they lean their head in to see the monitor or written copy, or hold the phone pinched between the ear and shoulder. A material handler may experience pain or injury as a result of looking down or to the side in the performance of a task such as picking up an item below the knees or off to the side. Once the invention is donned, the user will be warned upon excessive head tilt wherein when the user in the above examples incorrectly attempts to get closer to their monitor, or the material handler incorrectly attempts to bend over in order to lift or reach an item they are warned that their posture is out of neutral alignment giving them an opportunity to self-adjust to a correct posture.

Research leading to the present invention has found therapeutic benefit in posture training which utilizes a head mounted device to monitor inclination or tilt of the head in the saggital and coronal planes as indicative of posture of the upper body, including the head, neck, shoulders, and trunk, and to assertively remind the individual through visual feedback whenever his/her posture lapses to an incorrect position or condition. Furthermore, the effectiveness of MSD prevention and MSD injury prevention training is enhanced by optimizing individual and group awareness, and by allowing users to self-correct their behavior, over time. MSD training performance is elevated through use of the device, and by the device providing effective ongoing reinforcement of correct postures. The effect of the user's corrective action extends beyond the actual duration of the training or use session by creating within an individual a “muscle-kinesthetic memory” so that even when the device is not in use or activated, the individual actually “recalls” and retains proper head and body position to the benefit of related neck and back postures, this is also known as mind-body connection and or proprioception.

For those who seek to improve or overcome harmful postural behavior patterns or habits, a posture training program will benefit from visual biofeedback and assertive technical reinforcement to accelerate the process, and ensure more sustainable postural improvements. Conventional practice often relies heavily on verbal admonishments of others including management personnel, other professionals, and parents, and may require an extraordinary level of self-discipline and self-awareness, more than is reasonable to expect in many individuals. The present invention is an easy and structured method aimed at changing ingrained poor posture habits most individual's exhibit, in learning and implementing new correct postures, and in making correct postures second nature for all users.

A general lack of awareness that a person has poor posture is, perhaps, a primary factor why so many people fail to self-correct their own postures. Not only do most people fail to even consider their own postures but it is difficult to assess. In other words, most people don't know if their own posture is proper or not, nor do they know what change they should make to bring their own posture into a desirable range.

Accordingly, there is a need for a device that is able to determine correct posture and notify a user if their posture deviates from a normal, acceptable range. In this manner the device would ensure that correct posture is maintained.

Similarly, there is a need for a device that can help increase a person's awareness with regard to their own posture.

Additionally, there is a need to provide feedback to the user, in real time, whenever a significant-enough deviation from a normal predetermined range of acceptable posture occurs. Such feedback could be used to invite the user to correct their own posture whenever a significant-enough deviation from a normal range in posture occurs. In this way the user would, over the course of time, retrain their bodies into adopting proper posture.

Ideally, such a device could be used to detect head inclination in the saggital and coronal planes or deviation from the midline (vertical plane). The device could be attached to a hat, visor, safety hard helmet, eyeglasses, sunglasses, head band or it could be built-into (i.e., integrated into) a cap, hat, headset, headphone, earpiece, earphone, earbud, hairclip or other object worn by a person or included in various pieces of clothing such as bras, sport bras, belts, or other types of apparel and accessories.

Ideally, such a device could include a battery powered microcontroller, or other sensing device interfaced with the level sensor, monitors the user's head tilt and provides an LED indication or warning, whenever the user's head tilt exceeds a predetermined limit to the front, rear, left and right sides or intermediate angle. The use of a calibrated level sensor, enables the microcontroller to be configured to allow the user to preset the device for: individual's best neutral posture, optimal head tilt limits, and to select different modes, such as beginning or advance training, sitting or standing, and to set a training session duration during which the number and direction of head tilt deviations detected may be counted. Deviation data is displayed to the user on an LCD display which counts both cumulative and training session deviation totals, and which may be reset by the user or an organization's representative for identification, analysis, tracking and posture improvement purposes. The microcontroller may be specifically configured to recognize and filter out or ignore gross movements, such as looking down at the ground surface when walking, while still providing feedback to the user of finer postural deviations, as well as the amount of time during which poor posture is detected prior to activating the poor or negative posture correction indicator, such as 0.25 to 0.5 seconds.

Accordingly, there exists today a need for a Posture Correction Device that helps to ameliorate the above-mentioned problems and difficulties as well as ameliorate those additional problems and difficulties as may be recited in the “OBJECTS AND SUMMARY OF THE INVENTION” or discussed elsewhere in the specification or which may otherwise exist or occur and that are not specifically mentioned herein.

Clearly, such a device would be useful and desirable.

2. Description of Prior Art

Posture monitor and correction devices are, in general, known. For example, the following patent documents describe various types of these devices, some of which may have some degree of relevance to the invention. Other patent documents listed below may not have any significant relevance to the invention. The inclusion of these patent documents is not an admission that their teachings anticipate any aspect of the invention. Rather, their inclusion is intended to present a broad and diversified understanding regarding the current state of the art appertaining to either the field of the invention or possibly to other related or even distal fields of invention.
U.S. Pat. No. 5,425,378 to Swezey, et al., that issued on Jun. 20, 1995; and
U.S. Pat. No. 5,158,089 to Swezey, et al., that issued on Oct. 27, 1992.

While the structural arrangements of the above described devices may, at first appearance, have similarities with the present invention, they differ in material respects. These differences, which will be described in more detail hereinafter, are essential for the effective use of the invention and which admit of the advantages that are not available with the prior devices.

### OBJECTS AND SUMMARY OF THE INVENTION

It is an object of the present invention to provide a posture monitor and correction device for monitoring an individual's posture and identifying the incorrect ones with the aide of warning devices for improvement and training in the workplace or at home. The device which detects head inclination in the saggital and coronal planes or deviation from the midline (vertical plane) can be attached to a hat, visor, safety hard helmet or built in (integrated into) a cap or hat or into various pieces of clothing such as bras, sport bras and accessories. A battery powered microcontroller, or other sensing device interfaced with the level sensor, monitors the user's head tilts and provides an LED indication or warning whenever the user's head tilt and provides an LED indication or warning whenever the user's head exceeds preset limits to the front, rear, left and right sides. The use of a professional level sensor enables the microcontroller to be configured to allow the user to preset the device for: individual's best neutral posture, optimal head tilt limits, and to select different modes, such as beginning or advance training, sitting or standing, and to set a training session duration during which the number and direction of head tilt deviations detected may be counted. Deviation data is displayed to the user on an LCD display which counts both cumulative and training session deviation totals, and which may be reset by the user or an organization's representative for identification, analysis, tracking and posture improvement purposes. The microcontroller may be specifically configured to recognize and filter out or ignore gross movements, such as looking down at the ground surface when walking, while still providing feedback to the user of finer postural deviations, as well as the amount of time during which poor posture is detected prior to activating the poor or negative posture correction indicator, such as 0.25 to 0.5 seconds.

It is also an important object of the invention to provide a posture monitor and correction device that senses tilt of a wearer's head, provides an indication or warning to the wearer whenever the tilt reaches a predetermined deviation limits to the front, rear, left or right side, so as to enable the perception and maintenance of proper posture.

Still another object of the invention is to provide a posture monitor and correction device that can give a user capability of setting or detecting different limits at which tilt will be indicated.

Still yet another object of the invention is to provide a posture monitor and correction device that recognizes, registers and accumulates data related to identifying monitored improper postures, implements corrective measures and training based from the information gathered by the device.

Still yet another further object of the invention is to provide a posture monitor and correction device that detects head inclination in the saggital and coronal planes or deviation from the midline (vertical plane) and which can be attached to or integrated into a hat, visor, safety hard hat, safety helmet, eyeglasses, sunglasses, head band, cap, hat, headset, headphone, earpiece, earphone, earbud, hairclip or other object worn by a person including a bra, sport bra, belt, or other type of apparel or accessory.

Still yet one other further object of the invention is to provide a posture monitor and correction device that detects head inclination in the saggital and coronal planes or deviation from the midline (vertical plane) and wherein a first portion of the device can be attached to or integrated into a hat, visor, safety hard hat, safety helmet, eyeglasses, sunglasses, head band, cap, hat, headset, headphone, earpiece, earphone, earbud, hairclip or other object worn by a person including a bra, sport bra, belt, or other type of apparel or accessory, and wherein a second portion of the device can be attached to or integrated into a remaining object from the above list, and wherein the first and second portions of the device are able to cooperate with each other sufficient to operate.

Other objects and advantages of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

Briefly, a posture correction device that is constructed in accordance with the principles of the present invention includes a Posture Monitoring and Correction Unit that includes one or more Posture Monitoring and Correction Alert Warning Lights or other means for providing feedback to a user and which is attached to a Hat or Cap or other article of clothing or accessory worn by a person, and which includes a Posture Monitoring and Correction Control and Data Unit for the collection and transmission of data for remote processing and a Power Source Unit.

There has thus been outlined, rather broadly, some of the features of the invention in order that the detailed description thereof may be better understood, and in order that the present contribution to the art may be better appreciated. There are additional features of the invention that will be described hereinafter.

In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

Use of the present invention is divided into two categories. One use category is for industrial use, such as posture monitoring and warning, corrective posture training, or ergonomics and biomechanics, in the workplace. Material or manual handlers, equipment operators, and at-desk worker's postures are monitored and users are automatically reminded via an LED warning indicator system when their head tilt and body posture leaves a correct or neutral posture position. Incorrect or negative postures place individuals at a higher risk for a Musculoskeletal Disorders (MSD), and/or MSD injury or illness. The LED indicator and warning system alerts the user via alert warning lights located under a cap's bill or built into the bill of the cap, allowing the user to self-correct his/her posture by bringing their head back into a neutral position. Users are able to self-adjust their postures, and over a period of time negative postural habits are replaced with positive postures, or new habits. The second category is for home use and is directed towards personal and family use wherein wearers of backpacks, and users of electronic devices such as cellular phones and gaming devices wherein users of such devices display poor postures when they read, text, or talk on a phone. As technology advances personal devices are being used by more people, more often. As an example, the Y generation uses texting as a way of communicating, with many users texting over 100 times per day. Family members generally have no skilled knowledge of MSDs or anatomy, and have little control over their family's postures and biomechanics, and have no effective method of training or retraining family member's postures in such a way which promotes sustainable positive postural change. Young adults can be incentivized or rewarded by their parents, which reinforces behavioral-change and expedites the corrective and positive postural changes that are necessary for long-term health.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1: FIG. 1 is an upper perspective view of the present invention. This is a perspective view showing the Posture Monitoring and Correction Device.

FIG. 2: FIG. 2 is a left side view of the present invention. This is the left side view.

FIG. 3: FIG. 3 is a right side view of the present invention. This is the right side view.

FIG. 4: FIG. 4 is a front view of the present invention. This is the front side view.

FIG. 5: FIG. 5 is an end view of the present invention. This is the back side view.

FIG. 6: FIG. 6 is a top view of the present invention. This is the top side view.

FIG. 7: FIG. 7 is a bottom view of the present invention. This is the bottom side view.

FIG. 8: FIG. 8 is an alternative view of the present invention. This is the side to side tilt view.

FIG. 9: FIG. 9 is a first alternative view of the present invention. This is the front to back tilt view.

FIG. 10: FIG. 10 is a second alternative view of the present invention. This is the section cut view.

### DETAILED DESCRIPTION OF THE INVENTION

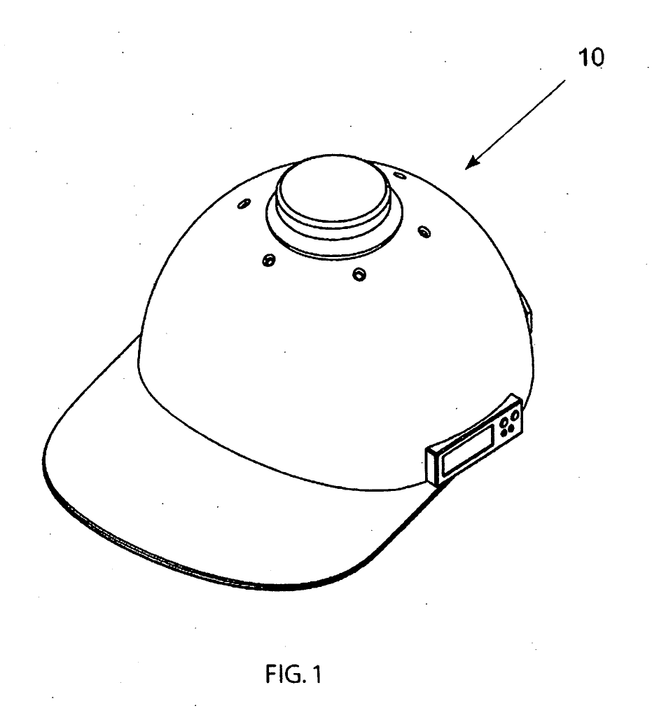

Referring on occasion to all of the FIGURE drawings and now, in particular to FIG. 1, is shown a posture monitoring and correction device, identified in general, by the reference numeral 10.

The reader will notice that reference is occasionally made throughout the DETAILED DESCRIPTION OF THE INVENTION suggesting that the reader refer to a particular drawing FIGURE. The suggestion is at times made when the introduction of a new element requires the reader to refer to a different drawing FIGURE than the one currently being viewed and also when the timely viewing of another drawing FIGURE is believed to significantly improve ease of reading or enhance understanding. To promote rapid understanding of the instant invention the reader is encouraged to periodically refer to and review each of the drawing FIGURES for possible cross-referencing of component parts and for other potentially useful information.

Certain examples are shown in the above-identified FIGURES and are described in greater detail below. In describing these examples, like or identical reference numerals may be used to identify common or similar elements.

Turning now descriptively to the drawings, in which similar reference characters denote similar elements throughout the several views, the figures illustrate the posture monitoring and correction device 10 that includes posture monitoring and correction alert warning lights (that include front LEDs 21 and side LEDs 22), a hat or cap 30, a posture monitoring and correction control and data unit 40 and a power source unit 50.

The posture monitoring and correction Unit 10 is a battery operated unit that can be programmed to monitor sense and measure the number of head movements in the forward, backward, left side and ride side (or in-between) direction from a level or neutral posture position. It can also be set to record the number of tilts accumulated during a predetermined amount of time, like in an hour, during the day or for the whole month. The unit 10 is attached to the upper portion of the baseball cap 30, where it is deemed the most sensible part and will not affect the weight of the unit 10, overall.

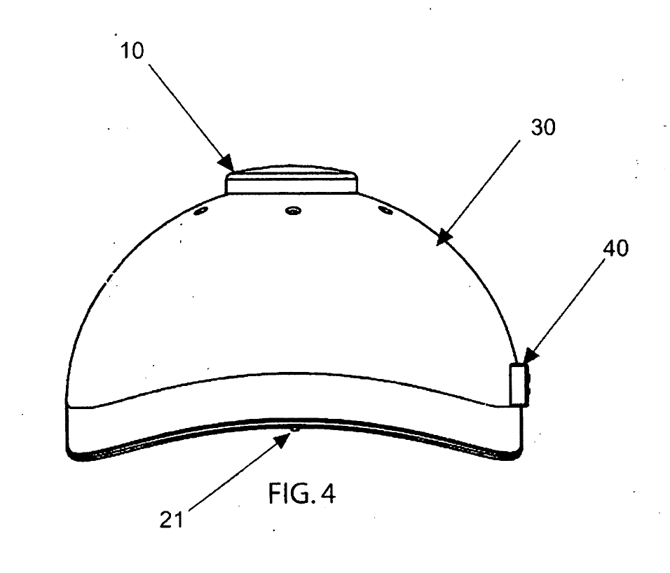

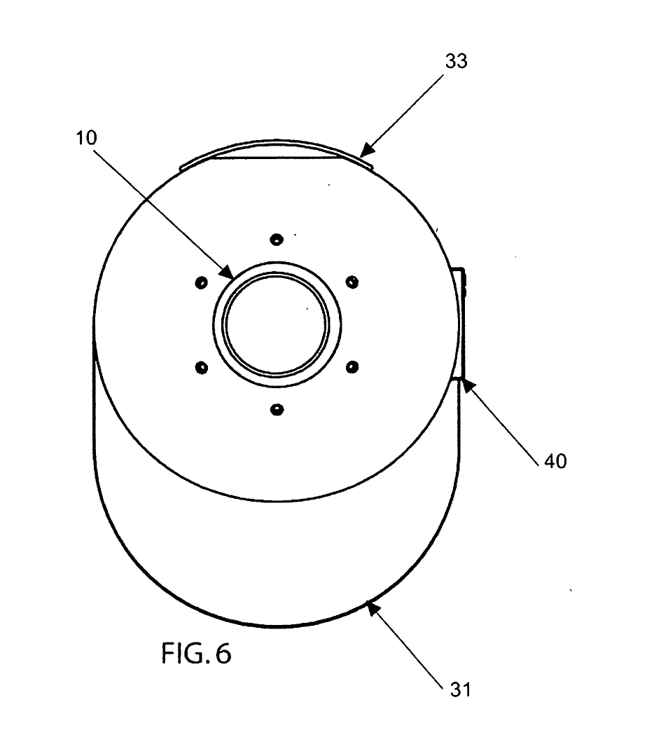

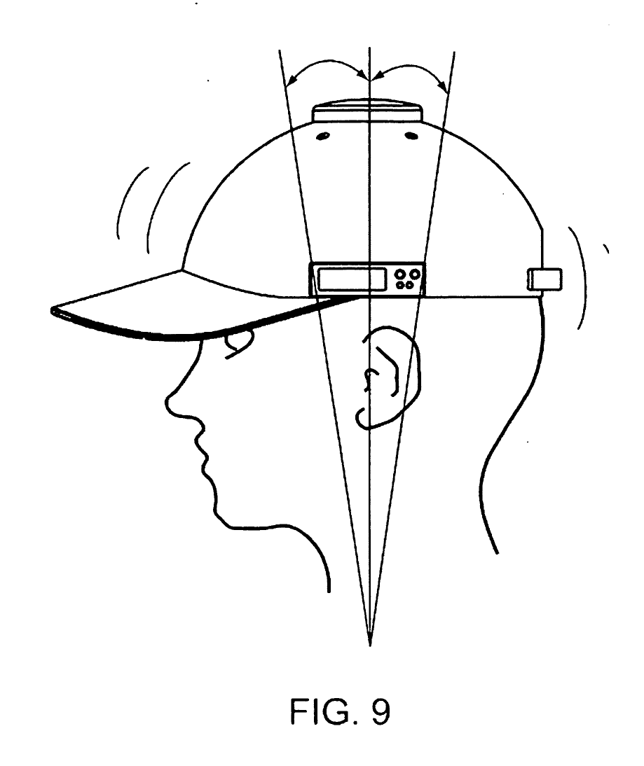

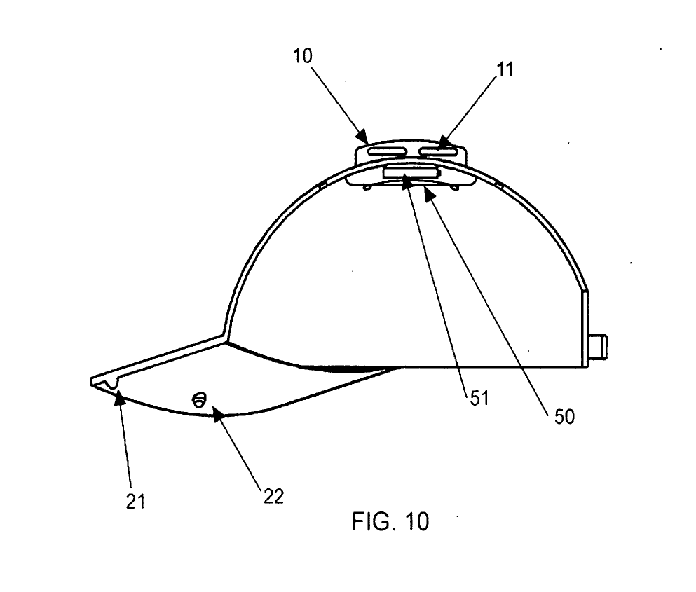

The monitor and correction unit 10 is attached to the upper portion of the cap 30 as illustrated in FIGS. 1 to 6 of the drawings. The monitor and correction unit 10 may be connected to the cap 30 through various conventional attachment methods commonly utilized in the industry. A level sensor 11 is the main component of the Monitoring and Correction unit 10. It is the proportional level sensor 11 that detects head inclination made in the saggital and coronal planes as shown FIGS. 9 and 10.

Alternatively, the posture monitoring and correction unit 10 can be positioned anywhere in the baseball cap 30 depending on its sensitivity to monitor and correct the tilt. Different variety of level sensing components can also be used to record the movements made by the user. These various elements can involve mercury switches, ball bearing contacts and accelerometers interfaced with the counters and timers to record the tilts.

The posture monitor and correction alert warning light functions as an indicator when a head tilt is made. The warning alert light affords the user/wearer an opportunity to acknowledge a bad posture has been made and makes an adjustment back to the neutral position. They are triggered and lights up every time a tilt is recorded in the front, back, left or right side direction. The warning alert would be in the form of an LED light however they may also be in audible or vibration electronic form. The lights 21, 22 are mounted underneath the brim of the hat or cap 30 and specifically in this embodiment, a baseball hat 30. The center light 21 would be at a minimum when located in the forward middle, underneath the brim of the hat 30 to monitor the forward and backward movements. Two additional lights 22, one on the left and one on the right, both located at the underside of the bill, serve as indicators when the user's head is tilted to the right or left out of neutral position. They would be activated upon reaching a certain degree of tilt and would stay lit, blink or stay lit for “x” amount of seconds until the user's head is brought back to the neutral posture.

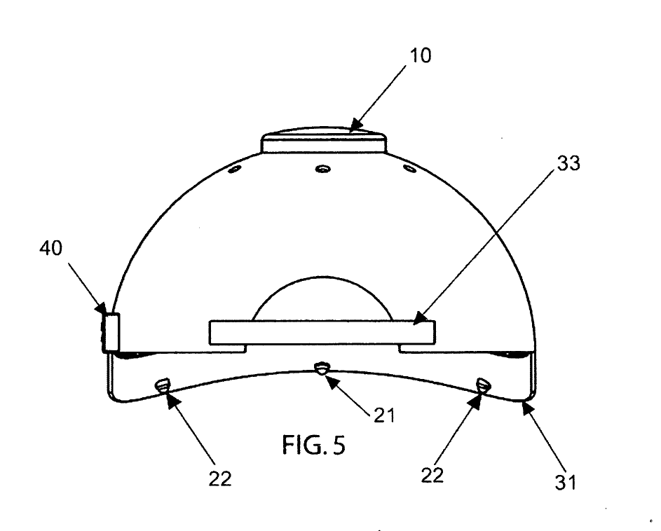

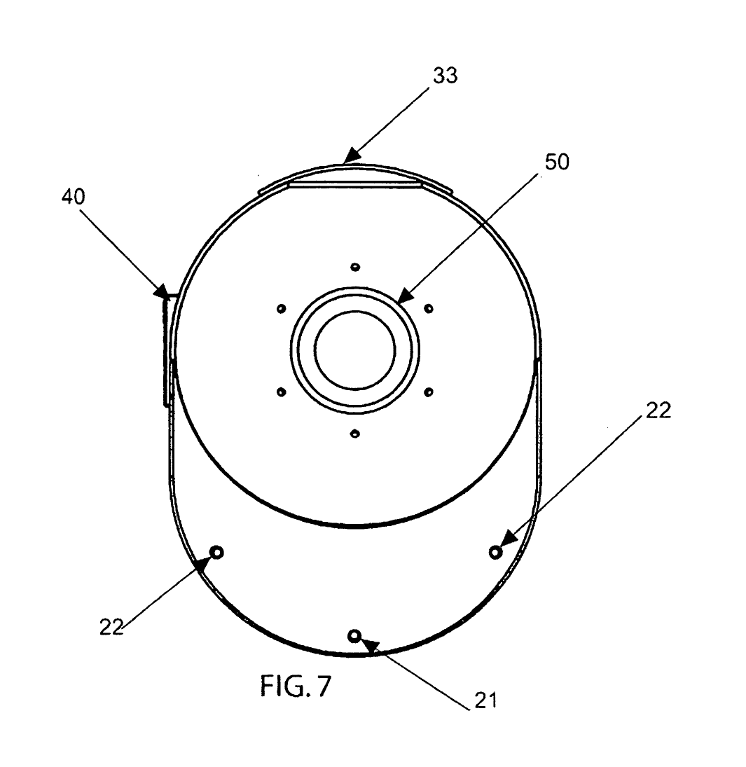

Thw posture monitoring and correction alert lights includes a pair of first and second side LEDs 22 disposed on opposite sides of an underside of a bill 31 (or brim) of the cap 30 and a front LED 21. The posture monitoring and correction alert lights are attached to an underneath portion of the bill 31 of the cap 30 as shown in FIGS. 5 and 7. The alert warning lights may be connected to the baseball cap 30 through conventional attachment methods. The alert warning lights are positioned in such a way that the movements can be distinguished in what position the tilt occurred be it in the front, back, left or right side. The front LEDs 21 and the side LEDs 22 are positioned underneath the brim 31 of the hat 30 as an indicator where the tilt was made. These Alert warning lights are triggered when the Monitoring and Correction unit 10 registers a tilt.

Alternatively, the alert warning lights can instead include a fiber optic light source with output under the bill 31 where desired or an audible or a vibratory component threaded through or attached to the baseball cap 30, that can be triggered when a tilt reaches the predetermined limit in the frontal, left and right side direction.

The Hat or Cap 30 specifically described herein as a baseball cap with the brim 31 that serves as a mount for both the Posture Monitoring and Correction Unit 10 at the upper portion area of the cap 30 and the Alert Warning Lights mounted under the brim 31 area of the cap 30. The device 10 ultimately would be designed to be a part of the hat or cap 30 or any headgear that can accommodate the parts of the device 10 and let them function the way they were designed.

The baseball cap 30 serves as a base to where the monitoring and correction components are mounted, namely monitoring and correction unit 10, the tilt warning lights, the control and data unit 40 and the battery unit 50 as shown in the Figures from 2 to 7.

Alternatively, the baseball cap 30 can be substituted with a head strap, hard hat, helmet, a head gear or piece of clothing such as bras, sports bras, belts and accessories to serve as a base for tilt monitoring and correction device 10. The material is not limited to cloth or plastic, metal can also be used as long as it is lightweight and will not add any burden to the user such as excessive compressive loading force.

The Posture Monitoring and Correction Control and Data Unit 40 having a display 41 and features to control the amount of tilt recorded and the duration they were recorded and is attached to the left side portion of the cap 30. The control and data unit 40 will include electronic counters with the information stored and compiled wherein the user can analyze the number of times their head tilted away from the neutral position. It will also have a tolerance level built in where certain movements are allowed without triggering the monitoring unit 40. The device 10 can be programmed to tabulate and document the number of tilts for a predetermined duration and will have the ability to retrieve that information if need be. The control and data unit 40 can also reset the counter to zero every time a new count has to be started. This data, gathered on a daily or cumulative basis, will be stored internally for tracking and study purposes. A feature is integrated to establish a personal neutral position or initial point of monitoring every time there is a new user. The initial point of monitoring can be established by the wearer standing flat against a wall, sitting up straight or comfortably and set a benchmark for their own personal starting neutral position.

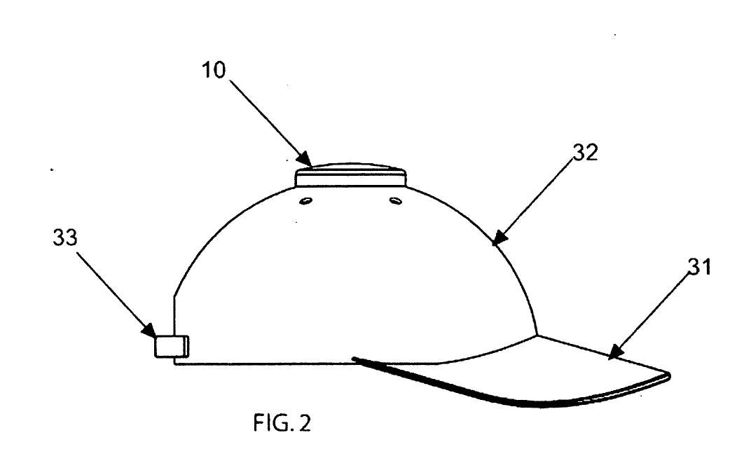

The Posture Monitoring and Correction control and data unit 40 turns on and off the monitoring and correction unit 10 through an On/Off switch 42 located in the frontal right area of the unit 40. The unit gathers data after setting the criteria through a Data and Time Switch 43 which toggles through a selection of time durations prior to recording the number of tilts. All the information and inputs are viewed and selected through a LCD Display 41. To start monitoring tilts, a neutral level position has to establish to serve as a basis for the degree of tilt. This is established by a Set and Start Switch 44 as well as initiating the recording of tilts. If it is necessary to start the count for whatever reason a Reset Switch 45 is also provided for just that. The control and data unit 40 is conveniently attached to the left side of the cap 30 for easy access as shown in FIG. 2. This unit 40 controls the whole device 10 and stores the data gathered as well. The data gathered can be downloaded electronically and remotely for further analysis.

Alternatively, the control and data unit 40 can be positioned on either side of the baseball hat 30, whatever is more convenient and more practical to the majority of users. The control and data unit 40 can also use Blue Tooth technology to transfer data to a central computer or server for further study and analysis.

The battery unit 50 having a compartment to house batteries 51 that powers the monitor unit 10, tilt warning lights and control and data unit 40. The battery unit 50 is attached to the upper area of the underside of the cap. The power source is designed to support the extended use of the unit's monitoring feature. The power source is also light weight to prevent added strain or discomfort to the user and can be replaced or recharged easily.

The battery unit 50 is connected to the inside top area of the cap 30. It is located opposite the monitoring and correction unit 10 which is attached to the top portion area of the baseball cap 30. It is attached adjacent to the monitoring and correction unit 10 to make it accessible for changing or recharging the batteries 51. The central location of the batteries 51 distributes and minimizes the weight in relation to the whole device.

Alternatively, the power source unit 50 can be comprised of watch batteries, rechargeable, disposable and new solar technology can be used instead of the 2 triple “A” batteries 51 in use for this embodiment.

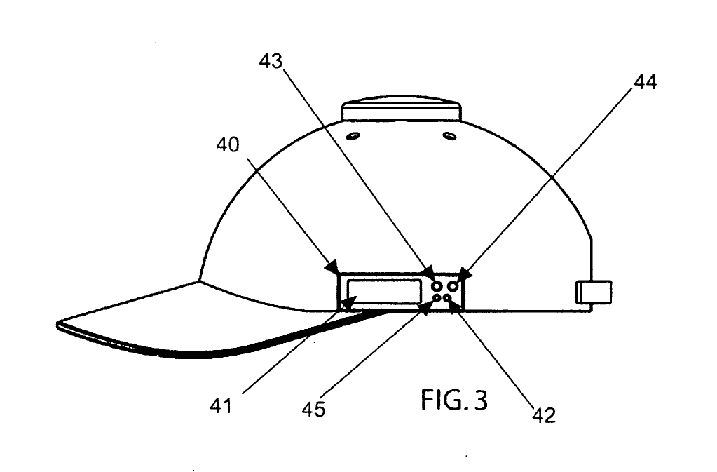

The baseball cap 30 is composed of the brim 31, a dome 32 and an adjustable strap 33. The brim 31 is attached to the frontal area and is an integral part of the baseball cap 30 and where the alert warning lights are mounted. The adjustable strap 33 as shown in FIG. 3 is attached to the back area of the baseball cap 30 to secure the monitoring and correction device 10 in place as well as to make it to fit to different head sizes.

The control and data unit 40 is connected electronically to all the monitoring device 10 parts except the baseball cap.

Alternatively, the Monitoring and Correction Device 10 can be used with hard hats, helmets, headgears, head bands, clothing such as bras, sports bras and accessories to mount and contain all the components.

To operate the Posture Monitoring and Correction Device 10, the user first activates the Posture Control and Data Unit 40 by manipulating the power switch 42 into the closed circuit as shown in FIG. 2 of the drawings. When the power switch 42 is turned on, the control and data unit 40 is in sync with the monitoring and correction unit 10, alert warning lights and the power source unit 50 thereby enabling the components to perform the way they were designed.

The use of the monitoring and correction device 10 starts by presetting the control and data unit 40 to the amount of time tilts have to be recorded. The hour/day/week/month switch, (Data and Time Switch 43) has to be activated and toggled to a selection where the tilt data accumulated will be recorded in an hour's span, day's span, week or month's span. A baseline neutral position has to be established for each user before any documentation of tilts can start. To create a neutral starting position, the user stands back against a wall, sits up in a comfortable upright position or orients oneself to a correct posture and with the Set and Start Switch 44 activated, a neutral level benchmark is established. And from that the device 10 is ready to monitor and correct unsafe tilt positions.

All conditions and parameters are displayed when inputted and can be viewed through the control and data unit's 40 LCD display 41. To re-start a count or change parameters for a count the Reset Switch 45 is available.

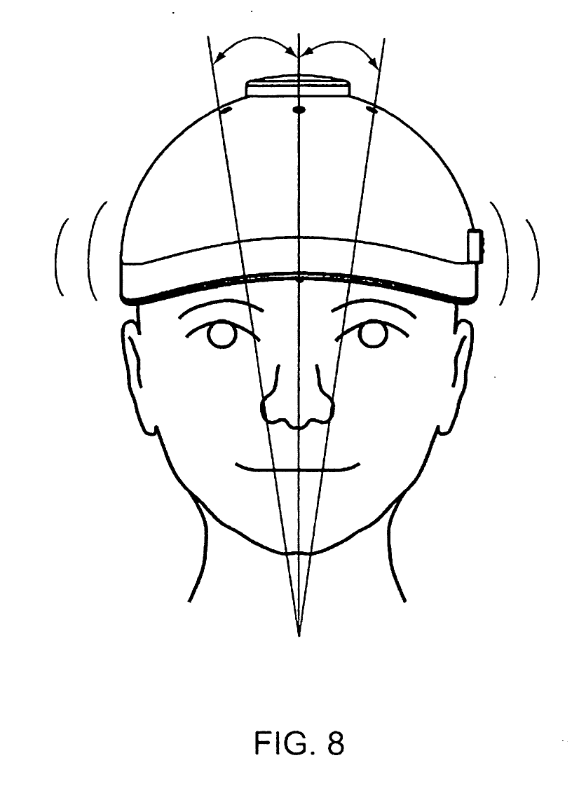

The monitoring and correction device 10 is mounted unto the user's head using the baseball cap 30 as shown in FIG. 8. The user will adjust the device 10 to a preset neutral starting point relative to the user's sitting position and placement of the baseball cap 30 on the head to initiate the count. The hat adjustment may be performed by moving the brim 31 higher or lower in relation to the line of sight or horizontal plane or by adjusting the strap 33 tighter or looser as shown in FIG. 3.

A front or back tilt movement as shown in FIG. 10 made more than the predetermined degree set in the monitoring and correction unit 10 activates the front LED 21 and registers in as a one count. A corrective counter movement in the opposite direction resets the lights and readies for the next tilt.

A left or right tilt movement as shown in FIG. 9 made more than the predetermined head tilt degree set in the monitoring and correction unit 10 activates either the right or left side LEDs 22, depending which side the tilt was made, registers in as a one count in the accumulation of data and will register in the control and data unit 40 as one count. A corrective counter movement in the opposite direction resets the lights for the next tilt.

The display 41 will show the current tilt count as well as the running total in each of the hour/day/week/month counter if the data and time switch 43 is toggled. Such data can be downloaded manually, digitally or remotely for further study and analysis.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect. It will be understood by those skilled in this art that other and further changes and modifications may be made without departing from the spirit and scope of the invention which is defined by the claims appended hereto.

What is claimed is:

1. A posture monitor and correction device, comprising:
(a) a level sensor attached to said device, said level sensor able to determine a deviation from level in any direction; and
(b) a control and data unit attached to said device that is in communication with said level sensor, wherein said control and data unit includes a microcomputer, and wherein said control and data unit is programmable to detect when said level sensor records a deviation in posture away from a normal posture, and create a log of deviations by accumulating one or more times that, within a predetermined period of time, said level sensor records said deviation in posture away from said normal posture; and wherein said control and data unit includes means for communicating said log of deviations to a recipient.

2. The posture monitor and correction device of claim 1 wherein said recipient includes a user of said device.

3. The posture monitor and correction device of claim 2 wherein said log is communicated to said user the moment said deviation is occurring.

4. The posture monitor and correction device of claim 3 including means for indicating a direction away from normal of said deviation that is attached to said device.

5. The posture monitor and correction device of claim 4 wherein said means for indicating a direction away from normal includes a light source that is illuminated.

6. The posture monitor and correction device of claim 5 wherein said light source includes a light-emitting-diode (LED).

7. The posture monitor and correction device of claim 4 wherein said means for indicating a direction away from normal includes an audible source attached to said device.

8. The posture monitor and correction device of claim 4 wherein said means for indicating a direction away from normal includes a vibrational source attached to said device.

9. The posture monitor and correction device of claim 1 wherein said recipient includes an electronic device.

10. The posture monitor and correction device of claim 9 wherein said electronic device is selected from the group consisting of a smart phone, a mobile computing platform, a computing tablet, and a computer.

11. The posture monitor and correction device of claim 9 wherein said electronic device includes means for presenting data from said log of deviations to said user or to another person.

12. The posture monitor and correction device of claim 9 wherein said electronic device includes means for analyzing said log of deviations and presenting data regarding any aspect of any of said deviations to said user or to another person.

13. The posture monitor and correction device of claim 1 wherein said control and data unit includes an electronic counter to record the number of times a user's head tilts sufficiently far away from a neutral position.

14. The posture monitor and correction device of claim 13 wherein said control and data unit includes a predetermined tolerance value, and wherein any tilting of said user's head that is less than said tolerance value is not recorded as a deviation from a normal posture.

15. The posture monitor and correction device of claim 1 including means for determining a neutral or normal posture.

16. The posture monitor and correction device of claim 15 wherein said means for determining a neutral posture includes calibrating said device whenever it is attached to said user and powered on.

17. The posture monitor and correction device of claim 16 wherein said means for determining a neutral posture includes said user standing flat against a wall, sitting up straight, or assuming a comfortable position to determine a personal neutral position and communicating attainment of said personal neutral position to said control and data unit, wherein said control and data unit records an output value provided by said level sensor as said neutral posture.

18. The posture monitor and correction device of claim 15 wherein said means for determining a neutral posture includes calibrating said device when it is first attached to a user and powered on, and wherein said device is able to save said neutral posture information for subsequent uses by said user.

19. The posture monitor and correction device of claim 1 wherein said device includes a display and wherein said display is able to indicate to said user information regarding said log of deviations.
